(19) United States
(12) Patent Application Publication
(10) Pub. No.: US 2005/0101943 A1
(43) Pub. Date: May 12, 2005

(54) Modular imbibition rate reducer for use with implantable osmotic pump

(75) Inventors: Rupal A. Ayer, Cupertino, CA (US); Scott D. Lautenbach, San Mateo, CA (US); Michael A. Desjardin, Sunnyvale, CA (US); Adrienne T. Higa, Los Angeles, CA (US)

Correspondence Address: Dewipat Incorporated, 4606 FM 1960 WEST, SUITE 400, HOUSTON, TX, 77069, US

(73) Assignee: ALZA Corporation

(21) Appl. No.: 10/978,597

(22) Filed: Nov. 1, 2004

Related U.S. Application Data
(60) Provisional application No. 60/518,111, filed on Nov. 6, 2003.

(51) Int. Cl. A61K 9/22

(52) U.S. Cl. 604/892.1

(57) ABSTRACT

An osmotic pump system includes a capsule having at least one delivery port, a membrane plug retained at an open end of the capsule remote from the delivery port, the membrane plug providing a fluid-permeable barrier between an interior and an exterior of the capsule, and a removable imbibition rate reducer attachable to the capsule. The imbibition rate reducer comprises one or more flow controllers selected from the group consisting of an orifice having a selected size smaller than a surface area of the membrane plug and a membrane having a selected thickness, surface area, radial compression, and permeability. The imbibition rate reducer allows customizable delivery of medicaments.

### CROSS-REFERENCE TO RELATED APPLICATIONS

This application claims the benefit of U.S. Provisional Application No. 60/518,111, filed Nov. 6, 2003.

### BACKGROUND OF THE INVENTION

The invention relates generally to implantable osmotic pumps for delivering beneficial agents. More specifically, the invention relates to an implantable osmotic pump having a semipermeable membrane for controlling the delivery rate of a beneficial agent.

Implantable osmotic pumps for delivering beneficial agents within the body of a patient are known in the art. For illustration purposes, FIG. 1 shows a cross-section of a typical implantable osmotic pump 100 having an implantable capsule 102. A delivery port 104 is formed at a closed end 106 of the capsule 102, and a semipermeable membrane plug 108 is received in an open end 110 of the capsule 102. The semipermeable membrane plug 108 forms a fluid-permeable barrier between the exterior and the interior of the capsule 102. A piston 112 is disposed in the capsule 102, forming two chambers 114, 116 within the capsule 102. The chamber 114 contains an osmotic agent 118, and the chamber 116 contains a beneficial agent 120. When the osmotic pump 100 is implanted in a patient, fluid from the body of the patient enters the chamber 114 through the semipermeable membrane plug 108, permeating the osmotic agent 118 and causing the osmotic agent 118 to swell. The swollen osmotic agent 118 pushes the piston 112 in a direction away from the semipermeable membrane plug 108, reducing the volume of the chamber 116 and forcing an amount of the beneficial agent 120 out of the capsule 102, through the delivery port 104, into the body of the patient.

The rate at which the osmotic pump 100 delivers the beneficial agent to the patient depends on the rate at which fluid is imbibed through the semipermeable membrane plug 108. The rate at which fluid is imbibed depends on the permeability, thickness, exposed surface area, and radial compression of the semipermeable membrane plug 108. Thus, once the osmotic pump 100 is assembled, the rate at which the beneficial agent 120 will be delivered to the patient is already established. This limits use of the osmotic pump in applications such as personalized care, where a caregiver requires the flexibility of administrating dosages to patients using non-standard dosing regimens. For these applications, the ability to adjust the delivery rate of the osmotic pump post-manufacture and pre-implantation could be beneficial. Preferably, the adjustment means does not have an adverse effect on the ability of the osmotic pump to deliver the beneficial agent.

### BRIEF SUMMARY OF THE INVENTION

In one aspect, the invention relates to an osmotic pump system which comprises a capsule having at least one delivery port, a membrane plug retained at an open end of the capsule remote from the delivery port, the membrane plug providing a fluid-permeable barrier between an interior and an exterior of the capsule, and a removable imbibition rate reducer attachable to the capsule. The imbibition rate reducer comprises one or more flow controllers selected from the group consisting of an orifice having a selected size smaller than a surface area of the membrane plug and a membrane having a selected thickness, surface area, radial compression, and permeability.

In another aspect, the invention relates to an osmotic pump system which comprises an implantable osmotic pump having a membrane plug at a first end and a delivery port at a second end remote from the first end. The membrane plug forms a fluid-permeable barrier between an interior and an exterior of the osmotic pump. The osmotic pump system further includes a removable imbibition rate reducer that is attachable to the osmotic pump. The imbibition rate reducer is selected from the group consisting of an orifice module having an orifice with a selected size, a membrane module having a membrane with a selected thickness, surface area, radial compression, and permeability, and combinations thereof. The orifice and membrane are configured to decrease an imbibition rate of the osmotic pump.

In another aspect, the invention relates to a method of adjusting a predefined delivery rate of an osmotic pump having a membrane plug forming a fluid-permeable barrier between an exterior and an interior of the osmotic pump. The method comprises reducing an imbibition rate of the osmotic pump by attaching an imbibition rate reducer to the osmotic pump so that fluid enters the membrane plug by passing through the imbibition rate reducer. The imbibition rate reducer comprises one or more flow controllers selected from the group consisting of an orifice having a selected size and a membrane having a selected thickness, surface area, radial compression, and permeability. The orifice is configured to reduce an effective surface area of the membrane plug, and the membrane is configured to increase an effective flow path length of the membrane plug.

In yet another aspect, the invention relates to an osmotic pump kit which comprises an implantable osmotic pump including a semipermeable membrane plug forming a fluid-permeable barrier between an interior and an exterior of the osmotic pump, a membrane module for increasing an effective flow path length of the membrane plug, and an orifice module for decreasing an effective surface area of the membrane plug, wherein the membrane module and orifice module are separately and independently attachable to or detachable from the osmotic pump.

Other features and advantages of the invention will be apparent from the following description.

### BRIEF DESCRIPTION OF THE DRAWINGS

FIG. 1 is a cross-section of a prior-art osmotic pump.

FIG. 2 is a cross-section of an orifice module for reducing imbibition rate of an osmotic pump according to one embodiment of the invention.

FIG. 3A shows a membrane module for reducing imbibition rate of an osmotic pump according to one embodiment of the invention.

FIG. 3B shows two membrane modules coupled together to form a membrane module stack according to another embodiment of the invention.

FIGS. 3C-3E show examples of possible modifications to the membrane module of FIG. 3A.

FIG. 3F shows an orifice module coupled to a membrane module for reduction of imbibition rate of an osmotic pump according to another embodiment of the invention.

FIG. 4A shows an osmotic pump system including a modular imbibition rate reducer installed on an osmotic pump in accordance with one embodiment of the invention.

FIG. 4B shows an osmotic pump system including a modular imbibition rate reducer installed on an osmotic pump in accordance with another embodiment of the invention.

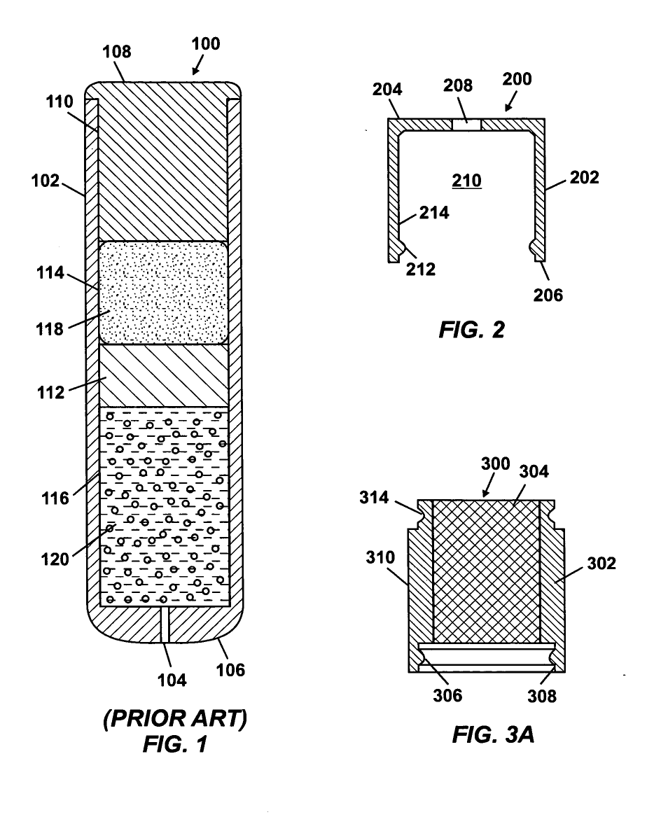

### DETAILED DESCRIPTION OF THE INVENTION

The invention will now be described in detail with reference to a few preferred embodiments, as illustrated in accompanying drawings. In the following description, numerous specific details are set forth in order to provide a thorough understanding of the invention. However, it will be apparent to one skilled in the art that the invention may be practiced without some or all of these specific details. In other instances, well-known features and/or process steps have not been described in detail in order to not unnecessarily obscure the invention. The features and advantages of the invention may be better understood with reference to the drawings and discussions that follow.

An imbibition rate reducer according to embodiments of the invention may be attached to or detached from an osmotic pump post-manufacture. When the imbibition rate reducer is attached to the osmotic pump, it functions to reduce the imbibition rate of the osmotic pump. In accordance with embodiments of the invention, the imbibition rate reducer includes an orifice to reduce the exposed surface area of a semipermeable membrane plug, which forms a fluid-permeable barrier between the exterior and interior of the osmotic pump, and/or one or more membranes to increase the effective flow path length of the membrane plug. The imbibition rate reducer allows the delivery rate of the osmotic pump to be reduced by an amount corresponding to the reduction in the imbibition rate of the osmotic pump. In one practical application, a caregiver could start with an osmotic pump designed to deliver a larger amount of medicament than what may be required for a particular patient. Based on the actual delivery rate desired by the caregiver, a reduction in exposed surface area and/or an increase in effective flow path length that would give the required imbibition rate can be determined and used to configure the imbibition rate reducer.

The imbibition rate reducer can be configured post-manufacture and pre-implantation using an orifice module and/or one or more membrane modules. For illustration purposes, FIG. 2 shows a cross-section of an orifice module 200 in accordance with one embodiment of the invention. The orifice module 200 includes a housing 202 having a capped end 204 and an open end 206. The open end 206 is sized to fit over an end portion of an osmotic pump (not shown). The capped end 204 is provided with an orifice 208 through which fluid can flow into the interior 210 of the housing 202. When the orifice module 200 is attached to the osmotic pump, the orifice 208 precedes the semipermeable membrane plug (not shown) of the osmotic pump. In this way, fluid from the exterior of the osmotic pump flows into the interior of the osmotic pump through the orifice 208 and the semipermeable membrane plug. The orifice 208 is sized such that it effectively reduces the exposed surface area of the semipermeable membrane plug, and hence the imbibition rate of the osmotic pump.

It should be noted that the invention is not limited to use of the single orifice 208 to control flow into the semipermeable membrane plug. For example, a cluster of holes can replace the single orifice 208, the combined flow area of the holes being selected to achieve the desired reduction in imbibition rate. Reduction in imbibition rate through the use of the orifice module 200 produces a corresponding reduction in the rate at which a beneficial agent is delivered by the osmotic pump.

The housing 202 is constructed so that it can be attached to an end portion of the osmotic pump including the semipermeable membrane plug. Preferably, the housing 202 can be snap-fitted to the osmotic pump. In one embodiment, an annular lip 212 is provided on an inner surface 214 of the housing 202. The annular lip 212 can engage with an annular groove (not shown) provided on an outer surface of the osmotic pump. Alternatively, the annular lip can be provided on the osmotic pump and the annular groove for engagement with the annular lip can be provided on the housing 202. Basically, any means of coupling tubular members, such as a threaded connection, can be used to affix the housing 202 to the osmotic pump. To maintain the osmotic pump in a sterile condition, the housing 202 should be attached to the osmotic pump using aseptic technique. In general, the cross-section of the housing 202 should be selected such that it can fit on or over an end portion of the osmotic pump. In general, any configuration such that a biofluidic path cannot be formed between the junction of the housing 202 and the end portion of the osmotic pump can be used. For example, if the end portion of the osmotic pump containing the semipermeable membrane plug has a circular cross-section, the housing 202 should preferably have a circular cross-section.

The housing 202 is formed from an inert and, preferably, biocompatible material. The material is “inert” in the sense that it will not react with the materials it will come in contact with during use. Exemplary inert, biocompatible materials include, but are not limited to, metals such as titanium, stainless steel, platinum and their alloys, and cobalt-chromium alloys and the like. Other compatible materials include polymers such as polyethylene, polypropylene, polycarbonate, polymethylmethacrylate (PMMA), and the like.

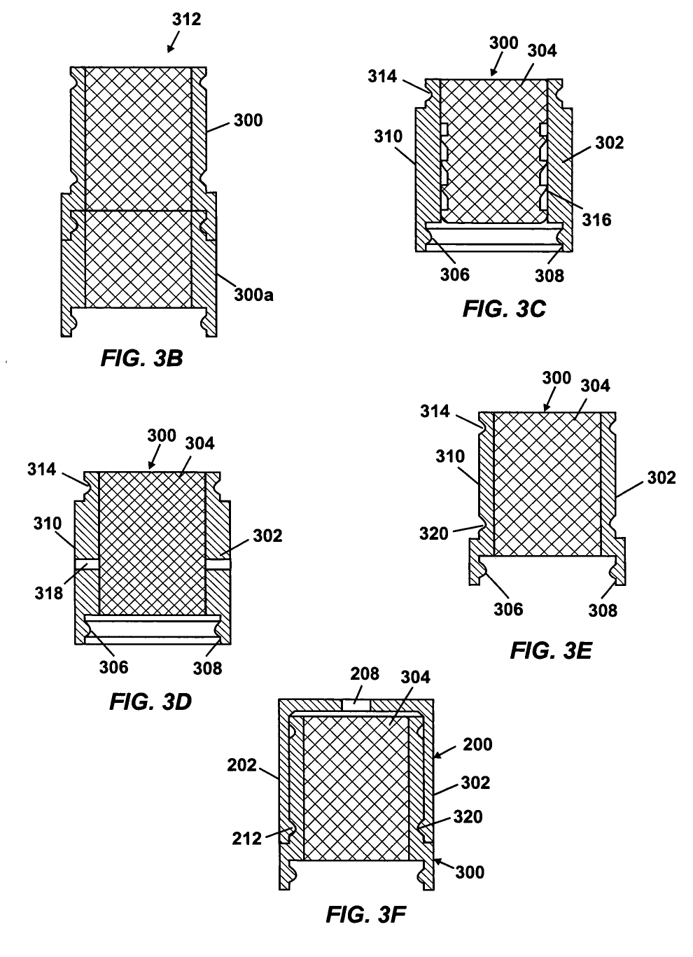

FIGS. 3A-3F show various embodiments of a membrane module. In FIG. 3A, a membrane module 300 includes a sleeve 302 and a membrane 304 inserted in the sleeve 302. The thickness of the membrane 304 is selected to increase the effective flow path length from the exterior of the osmotic pump (not shown), through the semipermeable membrane plug (not shown) at an end of the osmotic pump, to the interior of the osmotic pump. An increase in the effective flow path length produces a decrease in imbibition rate and a corresponding decrease in the delivery rate of the osmotic pump. The material used in making the membrane 304 may be the same as or may be different from the material used in making the semipermeable membrane plug of the osmotic pump. The material used in making the membrane 304 is preferably semipermeable and preferably can conform to the inner shape of the sleeve 302 upon wetting and adhere to the inner surface of the sleeve 302. Suitable semipermeable materials are typically polymeric materials, including, but not limited to, plasticized cellulosic materials, enhanced PMMAs such as hydroxyethylmethacrylate (HEMA), and elastomeric materials such as polyurethanes and polyamides, polyether-polyamind copolymers, thermoplastic copolyesters, and the like.

The exposed surface area of the membrane 304 may be the same as or may be different from the exposed surface area of the semipermeable membrane plug of the osmotic pump. That is, fluid imbibition can be controlled not just by the thickness of the membrane 304 but also by the exposed surface area of the membrane 304. The sleeve 302 radially constrains the membrane 304, exerting an amount of radial compression on the membrane 304. This radial compression along with the thickness, permeability, and exposed surface area of the membrane 304 can be selected to achieve a desired reduction in imbibition rate of the osmotic pump.

The membrane module 300 is constructed so that it can be attached to the osmotic pump post-manufacture and pre-implantation. Preferably, the membrane module 300 can be snap-fitted to the osmotic pump. In one embodiment, this could be accomplished by providing an annular lip 306 on an inner surface 308 of the sleeve 304 that can engage with an annular groove (not shown) on an end portion of the osmotic pump containing the semipermeable membrane plug. Alternatively, the annular lip could be provided on the osmotic pump and an annular groove that can engage with the annular lip can be provided on the sleeve 304. However, the invention is not limited to use of annular lip/annular groove to couple the membrane module 300 to the osmotic pump. In general, any means of coupling tubular members, such as a threaded connection, can be used to affix the membrane module 300 to the osmotic pump. Preferably, any coupling configuration used is such that a biofluidic path cannot be formed between the junction of the sleeve 302 and the end portion of the osmotic pump. The membrane module 300 should be attached to the osmotic pump using aseptic technique.

The membrane module 300 is also constructed so that a plurality of the membrane modules can be coupled together to form a membrane stack. In FIG. 3B, for example, a membrane stack 312 is formed by connecting the membrane modules 300, 300a. Note that the characteristics of the membrane modules in the stack, such as the thickness, permeability, exposed surface area, and radial compression of the membranes in the modules, can be the same or different. Returning to FIG. 3A, in one embodiment, an annular groove 314 is provided on the outer surface 310 of the membrane module 300 for engagement with an annular lip (similar to annular lip 306) on the inner surface of another membrane module, thereby allowing multiple membrane modules to be stacked to provide a desired flow path length. Other means of connecting tubular members, such as threaded connections, may also be employed to couple multiple membrane modules together. Preferably, any coupling configuration used is such that a biofluidic path cannot be formed between the junctions of multiple sleeves 302. The outer diameter of the sleeve 302 can be selected such that the outer surface 310 of the sleeve 302 is flush with the outer surface of the osmotic pump when the membrane module 300 is fitted to the osmotic pump.

The sleeve 302 is formed from an inert and, preferably, biocompatible material. Exemplary inert, biocompatible materials include, but are not limited to, metals such as titanium, stainless steel, platinum and their alloys, and cobalt-chromium alloys and the like. Other examples of compatible materials include polymers such as polyethylene, polypropylene, polycarbonate, polymethylmethacrylate (PMMA), and the like.

The membrane module 300 can be modified in various ways. For example, as shown in FIG. 3C, the outer surface of the membrane 304 could include ribs 316 (or threads, ridges, and the like) which form a seal between the membrane 304 and the sleeve 302. In FIG. 3D, the sleeve 302 includes holes 318 through which fluid can flow into the sleeve 302 or pressure can be vented out of the sleeve 302. The holes 318 can also double up as retention means for the membrane 304, as taught by Rupal Ayer in U.S. Pat. No. 6,270,787. In FIG. 3E, the sleeve 302 includes a mating surface, such as an annular groove 320, for engagement with a corresponding mating surface, such as the annular lip (212 in FIG. 2), on the orifice module (200 in FIG. 2). As shown in FIG. 3F, the annular lip 212 on the housing 202 of the orifice module 200 is fitted into the annular groove 320 on the sleeve 302 of the membrane module 300. When this structure is installed on an osmotic pump, the imbibition rate of the osmotic pump can be reduced by both the orifice 208 in the orifice module 200 and the membrane 304 in the membrane module 300.

In practice, an imbibition rate reducer can be constructed using any of the modular structures described in FIGS. 2 and 3A-3F. As described above, the orifice module and membrane module are designed such that they can be separately and independently attached to the osmotic pump. Additionally, a stack of membrane modules can be formed and attached to the osmotic pump. Also, the orifice module can be coupled to a membrane module, which can then be attached to the osmotic pump. The imbibition rate reducer can be installed on the osmotic pump post-manufacture and pre-implantation to reduce the imbibition rate of the osmotic pump by a selected amount, where a reduction in imbibition rate produces a corresponding reduction in the delivery rate of the osmotic pump.

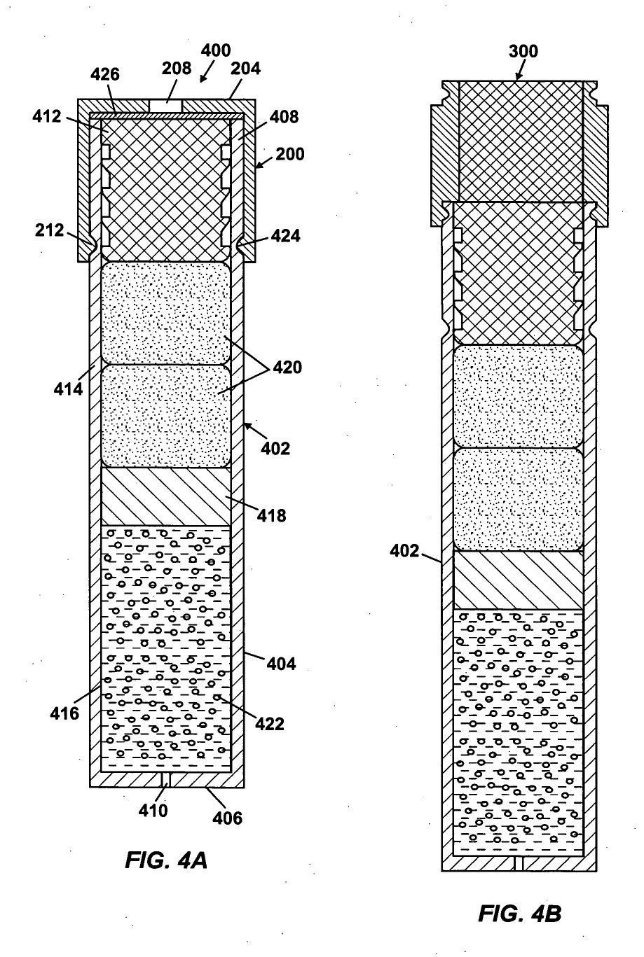

For illustration purposes, FIG. 4A shows an osmotic pump system 400 having an imbibition rate reducer, e.g., the orifice module 200, installed on an osmotic pump 402 according to an embodiment of the invention. The internal structure of the osmotic pump 402 is presented for illustration purposes only and is not to be construed as limiting the present invention. The present invention is generally applicable to all osmotic pumps having any number of shapes, and to all such pumps administered in implantable osmotic delivery techniques.

The osmotic pump 402, as illustrated in FIG. 4A, includes an elongated cylindrical capsule 404. The capsule 404 may be sized such that it can be implanted within a body. In FIG. 4A, one end 406 of the capsule 404 is closed and the other end 408 of the capsule 404 is open. The closed end 406 includes a delivery port 410. In an alternative embodiment, the closed end 406 may be modified to include a flow modulator (not shown), such as taught by Peterson et al. in U.S. Pat. No. 6,524,305. A semipermeable membrane plug 412 is received in the open end 408 of the capsule 404. The semipermeable membrane plug 412 may be inserted partially or fully into the open end 408. In the former case, the semipermeable membrane plug 412 may include an enlarged end portion that acts as a stop member engaging an end of the capsule 404. The outer surface of the semipermeable membrane plug 412 may have ribs, threads, ridges and the like which form a seal between the membrane 412 and the inner surface of the capsule 404, as taught by Chen et al. in U.S. Pat. No. 6,113,938.

The semipermeable membrane plug 412 is made of a semipermeable material that allows water to pass from an exterior of the capsule 404 into the interior of the capsule 404 while preventing compositions within the capsule from passing out of the capsule. Semipermeable materials suitable for use in the invention are well known in the art. Semipermeable materials for the membrane plug are those that can conform to the shape of the capsule upon wetting and that can adhere to the inner surface of the capsule. Typically, these materials are polymeric materials, which can be selected based on the pumping rates and system configuration requirements, and include, but are not limited to, plasticized cellulosic materials, enhanced PMMAs such as hydroxyethylmethacrylate (HEMA), and elastomeric materials such as polyurethanes and polyamides, polyether-polyamind copolymers, thermoplastic copolyesters, and the like.

Two chambers 414, 416 are defined inside the capsule 404. The chambers 414, 416 are separated by a partition 418, such as a slidable piston or flexible diaphragm, which is configured to fit within the capsule 404 in a sealing manner and to move longitudinally within the capsule. Preferably, the partition 418 is formed from an impermeable resilient material. As an example, the partition 418 may be a slidable piston made of an impermeable resilient material and including annular ring shape protrusions that form a seal with the inner surface of the capsule 404. An osmotic agent 420 is disposed in the chamber 414 adjacent the semipermeable membrane plug 412, and a beneficial agent 422 to be delivered to a body is disposed in the chamber 416 adjacent the delivery port 410. The partition 418 isolates the beneficial agent 422 from the environmental liquids that are permitted to enter the capsule 404 through the semipermeable membrane plug 412 such that in use, at steady-state flow, the beneficial agent 422 is expelled through the delivery port 410 at a rate corresponding to the rate at which liquid from the environment of use flows into the osmotic agent 420 through the orifice module 200 and semipermeable membrane plug 412.

The osmotic agent 420 may be in the form of tablets as shown or may have other shape, texture, density, and consistency. For example, the osmotic agent 420 may be in powder or granular form. The osmotic agent may be, for example, a nonvolatile water soluble osmagent, an osmopolymer which swells on contact with water, or a mixture of the two.

In general, the present invention applies to the administration of beneficial agents, which include any physiologically or pharmacologically active substance. The beneficial agent 422 may be any of the agents which are known to be delivered to the body of a human or an animal such as medicaments, vitamins, nutrients, or the like. Drug agents which may be delivered by the present invention include drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, anti-inflammatory corticosteroids, ocular drugs and synthetic analogs of these species. An exemplary list of drugs that may be delivered using the osmotic pump system 400 is disclosed in U.S. Pat. No. 6,270,787. The list is incorporated herein by reference.

The beneficial agent 422 can be present in a wide variety of chemical and physical forms, such as solids, liquids and slurries. On the molecular level, the various forms may include uncharged molecules, molecular complexes, and pharmaceutically acceptable acid addition and base addition salts such as hydrochlorides, hydrobromides, sulfate, laurylate, oleate, and salicylate. For acidic compounds, salts of metals, amines or organic cations may be used. Derivatives such as esters, ethers and amides can also be used. A beneficial agent can be used alone or mixed with other beneficial agents. The beneficial agent may optionally include pharmaceutically acceptable carriers and/or additional ingredients such as antioxidants, stabilizing agents, permeation enhancers, etc.

Materials which may be used for the capsule 404 must be sufficiently rigid to withstand expansion of the osmotic agent 420 without changing its size or shape. Further, the materials should ensure that the capsule 404 will not leak, crack, break, or distort under stress to which it could be subjected during implantation or under stresses due to the pressures generated during operation. The capsule 404 may be formed of chemically inert biocompatible, natural or synthetic materials which are known in the art. The capsule material is preferably a non-bioerodible material which remains in the patient after use, such as titanium. However, the material of the capsule may alternatively be a bioerodible material which bioerodes in the environment after dispensing of the beneficial agent. Generally, preferred materials for the capsule 404 are those acceptable for human implantation.

In general, typical materials of construction suitable for the capsule 404 according to the present invention include non-reactive polymers or biocompatible metals or alloys. The polymers include acrylonitrile polymers such as acrylonitrile-butadiene-styrene terpolymer, and the like; halogenated polymers such as polytetraflouroethylene, polychlorotrifluoroethylene, copolymer tetrafluoroethylene and hexafluoropropylene; polyimide; polysulfone; polycarbonate; polyethylene; polypropylene; polyvinylchloride-acrylic copolymer; polycarbonate-acrylonitrile-butadiene-styrene; polystyrene; and the like. Metallic materials useful for the capsule 404 include stainless steel, titanium, platinum, tantalum, gold, and their alloys, as well as gold-plated ferrous alloys, platinum-plated ferrous alloys, cobalt-chromium alloys and titanium nitride coated stainless steel.

A capsule 404 made from the titanium or a titanium alloy having greater than 60%, often greater than 85% titanium, is particularly preferred for the most size-critical applications, for high payload capability and for long duration applications, and for those applications where the formulation is sensitive to body chemistry at the implantation site or where the body is sensitive to the formulation. In certain embodiments, and for applications other than the fluid-imbibing devices specifically described, where unstable beneficial agent formulations are in the capsule 404, particularly protein and/or peptide formulations, the metallic components to which the formulation is exposed must be formed of titanium or its alloys as described above.

The orifice module 200 is installed by, for example, snapping the annular lip 212 on the housing 202 into an annular groove 424 on the outer surface of the capsule 404. As previously mentioned, other means of installing the orifice module 200 may be used, such as a threaded connection. An optional porous substrate 426, such as a screen or mesh, may be inserted between the orifice 208 and the semipermeable membrane plug 412 to prevent deformation of the membrane 412. That is, the semipermeable membrane plug 412 can bulge out because of pressure inside the capsule 404. The semipermeable membrane plug 412 may extend into the orifice 208 if the bulging is not controlled. If desired, the housing 202 may be sized such that a chamber (not shown) is formed between the semipermeable membrane plug 412 and the capped end 204 of the housing 202 that allows for a degree of movement of the semipermeable 412 into the housing 202 as a result of pressure in the interior of the capsule 404. The capped end 204 can act as a stopper to prevent the semipermeable membrane plug 412 from being separated from the osmotic pump 402.

FIG. 4B shows the membrane module 300 installed on the osmotic pump 402. As previously mentioned, any of the disclosed orifice module (200 in FIG. 2) and membrane modules (300 in FIGS. 3A-3D) and other variations may be installed on an osmotic pump to reduce the imbibition rate of the osmotic pump by a selected amount.

The invention typically provides the following advantages. The invention provides a means of adjusting the delivery rate of an osmotic pump post-manufacture. A variety of delivery profiles can be achieved without adversely affecting the operation of the osmotic pump. This gives caregivers flexibility in treatment options.

While the invention has been described with respect to a limited number of embodiments, those skilled in the art, having benefit of this disclosure, will appreciate that other embodiments can be devised which do not depart from the scope of the invention as disclosed herein.

1. An osmotic pump system, comprising:
a capsule having at least one delivery port;
a membrane plug retained at an open end of the capsule remote from the delivery port, the membrane plug providing a fluid-permeable barrier between an interior and an exterior of the capsule; and
a removable imbibition rate reducer attachable to the capsule, the imbibition rate reducer comprising one or more flow controllers selected from the group consisting of an orifice having a selected size smaller than a surface area of the membrane plug and a membrane having a selected thickness, surface area, radial compression, and permeability.

2. The osmotic pump system of claim 1, wherein the orifice is formed in a capped end of a housing and the membrane is radially constrained by a sleeve, the housing and the sleeve having mating surfaces that can be separately and independently engaged with or disengaged from a corresponding mating surface on the capsule.

3. The osmotic pump system of claim 2, wherein the sleeve further includes a mating surface that can be engaged with or disengaged from a corresponding mating surface on another sleeve enclosing a membrane so as to allow a stack of membranes to be formed.

4. The osmotic pump system of claim 2, wherein the sleeve further includes a mating surface that can be engaged with or disengaged from a corresponding mating surface on the housing so as to allow the sleeve to be coupled to or decoupled from the housing.

5. The osmotic pump system of claim 2, wherein the membrane has a plurality of spaced protrusions formed on an outer surface thereof that can be engaged with or disengaged from an inner surface of the sleeve.

6. The osmotic pump system of claim 2, wherein the sleeve includes one or more holes allowing communication between the interior and the exterior of the sleeve.

7. The osmotic pump system of claim 1, further comprising a first and a second chamber defined in the capsule for containing an osmotic agent and a beneficial agent, respectively.

8. The osmotic pump system of claim 7, further comprising a movable partition disposed between the first and second chambers.

9. The osmotic pump system of claim 1, further comprising a porous substrate that can be inserted between the orifice and the membrane plug or between the orifice and the membrane to prevent deformity of the membrane plug or membrane.

10. An osmotic pump system, comprising:
an implantable osmotic pump having a membrane plug at first end and a delivery port at a second end remote from the first end, the membrane plug forming a fluid-permeable barrier between an interior and an exterior of the osmotic pump; and
a removable imbibition rate reducer attachable to the osmotic pump, the imbibition rate reducer being selected from the group consisting of an orifice module having an orifice with a selected size, a membrane module having a membrane with a selected thickness, surface area, radial compression, and permeability, and combinations thereof;
wherein the orifice and membrane are configured to decrease an imbibition rate of the osmotic pump by a selected amount.

11. The osmotic pump system of claim 10, wherein the orifice module and the membrane module can be separately and independently engaged with or disengaged from the osmotic pump.

12. The osmotic pump of claim 10, wherein a plurality of the membrane module can be removably attached to form a stack of membranes that can be removably attached to the osmotic pump.

13. The osmotic pump of claim 11, wherein the orifice module can be removably attached to the membrane module.

14. The osmotic pump of claim 13, further comprising a porous substrate for insertion between the membrane module and the orifice module so as to prevent deformity of the membrane in the membrane module.

15. The osmotic pump of claim 10, further comprising a porous substrate for insertion between the membrane plug and the orifice module so as to prevent deformity of the membrane plug.

16. A method of adjusting a predefined delivery rate of an osmotic pump having a membrane plug forming a fluid-permeable barrier between an exterior and an interior of the osmotic pump, comprising:
reducing an imbibition rate of the osmotic pump by attaching an imbibition rate reducer to the osmotic pump so that fluid enters the membrane plug through the imbibition rate reducer;
wherein the imbibition rate reducer comprises one or more flow controllers selected from the group consisting of an orifice having a selected size and a membrane having a selected thickness, surface area, radial compression, and permeability;
wherein the orifice is configured to reduce an effective surface area of the membrane plug and the membrane is configured to increase an effective flow path length of the membrane plug.

17. An osmotic pump kit, comprising:
an implantable osmotic pump including a semipermeable membrane plug forming a fluid-permeable barrier between an interior and an exterior of the osmotic pump;
a membrane module for increasing an effective flow path length of the membrane plug;
an orifice module for decreasing an effective surface area of the membrane plug;
wherein the membrane module and orifice module are separately and independently attachable to and removable from the osmotic pump.

18. The osmotic pump kit of embodiment 17, further comprising a porous substrate that can be inserted between the membrane plug and the orifice module or between the orifice module and the membrane module to prevent deformity of the membrane plug or the membrane module, respectively.
